(19) United States
(12) Patent Application Publication
(10) Pub. No.: US 2026/0263777 A1
(43) Pub. Date: Sep. 10, 2026

(54) Extracorporeal Magnetically Levitated Blood Pump

(71) Applicant: MAGASSIST CO., LTD., Suzhou, Jiangsu (CN)

(72) Inventors: Renmu TAN, Suzhou, Jiangsu (CN); Ifan YEN, Suzhou, Jiangsu (CN); Polin HSU, Suzhou, Jiangsu (CN)

(73) Assignee: MAGASSIST CO., LTD., Suzhou, Jiangsu (CN)

(21) Appl. No.: 18/871,806

(22) PCT Filed: Jun. 1, 2023
(86) PCT No.: PCT/CN2023/097903

§ 371 Date: Dec. 4, 2024

(30) Foreign Application Priority Data
Jun. 6, 2022 (CN) 202210635919.2

(51) Int. Cl. A61M 60/232; A61M 1/36; A61M 60/113; A61M 60/117; A61M 60/419; A61M 60/81

(52) U.S. Cl. CPC A61M 60/232; A61M 60/113; A61M 60/117; A61M 60/419; A61M 60/81; A61M 1/367; A61M 2205/103

(57) ABSTRACT

Some embodiments of the present disclosure provide an extracorporeal magnetically levitated blood pump with improved stiffness of a rotor, including a motor and a pump head operably connected to the motor. The pump head includes a pump housing and an impeller which is accommodated in the pump housing and is configured to levitate in the pump housing and driven by the motor to rotate, the impeller includes an impeller housing and blades disposed on the impeller housing, a rotor is disposed in the impeller housing, and the axial height of the rotor is 7.86-10.34 mm, further 8.56-9.87 mm, and further 8.834-9.537 mm.

### CROSS-REFERENCE TO RELATED APPLICATIONS

The present disclosure is a national stage application of International Patent Application No. PCT/CN2023/097903, which is filed on Jun. 1, 2023, and claims priority to Chinese Patent Application No. 202210635919.2, filed on Jun. 6, 2022 and entitled “Extracorporeal Magnetically Levitated Blood Pump”, the contents of which are hereby incorporated by reference in its entirety.

### TECHNICAL FIELD

The present disclosure relates generally to an extracorporeal magnetically levitated blood pump.

### BACKGROUND

Blood pumps are classified into implantable type, extracorporeal type and interventional type, and centrifugal pump technology is mainly used for implantable and extracorporeal blood pumps. After evolution of three generations of technology, a third-generation magnetically levitated blood pump is recognized as the pump with best blood compatibility. A magnetically levitated blood pump adopts a magnetically levitated system, and an impeller in the pump levitates in a pump housing, so that blood cells are prevented from being damaged due to friction of a mechanical bearing, and better blood compatibility is obtained. The design of a blood pump is usually a balance between blood compatibility and hydraulic performance, and the factors that affect the blood compatibility are shear stress the blood is subjected to and duration of exposure in the shear stress.

The design of the existing implantable blood pump mostly aims at ventricular assistance, which lacks expandability. The extracorporeal blood pump can be added with a membrane lung to be expanded into an Extracorporeal Membrane Oxygenation (ECMO) system, and even can be applied to a cardiopulmonary bypass machine in a cardiac operation if having high hydraulic performance.

For an implantable blood pump capable of realizing extended application, the operational stability requirement of the blood pump is higher, and blood compatibility comparable or even higher than that of ventricular assistance application is required. At present, most of implantable blood pumps suitable for ECMO adopt a mechanical bearing, and only one company in the United States adopts extracorporeal magnetically levitated blood pump technology. Most cardiopulmonary bypass machines use large peristaltic pumps with low blood compatibility.

### SUMMARY

In view of the above defects, an objective of the present disclosure is to design an extracorporeal magnetically levitated blood pump with improved rotor stiffness, to improve operation stability of the blood pump, so as to be suitable for a ventricular assistance system, an ECMO system and even a cardiopulmonary bypass machine.

The extracorporeal magnetically levitated blood pump of the present disclosure includes a motor and a pump head operably connected to the motor. The pump head includes a pump housing and an impeller which is accommodated in the pump housing and is configured to levitate in the pump housing and driven by the motor to rotate, and the impeller includes an impeller housing and blades disposed on the impeller housing, and a rotor is disposed in the impeller housing. An axial height of the rotor is 7.86-10.34 mm, further 8.56-9.87 mm, and further 8.834-9.537 mm.

For the extracorporeal magnetically levitated blood pump of the present disclosure, by reducing overall height of the rotor, the coupling area of active and passive magnets is reduced, thereby reducing influence of magnetic coupling effect between the active and passive magnets on stiffness of the rotor, improving the stiffness of the rotor, and further improving operational stability.

In addition, by improving a balancing ring structure to form a radial bulge part, an original straight ring is changed to an L-shaped ring or L-shape-like ring, so that moment of inertia of the rotor is increased, the stiffness of the rotor is improved, then the stiffness of the rotor is improved in a physical or mechanical perspective, and the operational stability is improved.

### BRIEF DESCRIPTION OF THE DRAWINGS

FIG. 1 is a structure diagram of a perspective view of an extracorporeal magnetically levitated blood pump provided by an embodiment of the present disclosure.

FIG. 2 is a schematic diagram of a top view of a motor (with a motor housing removed) in FIG. 1.

FIG. 3 is a schematic diagram of a sectional view taken along A-A of FIG. 2.

FIG. 4 is a schematic diagram of a sectional view of a pump head of FIG. 1.

FIG. 5 is a partial structure diagram of FIG. 4.

FIG. 6 is an enlarged partial schematic diagram of FIG. 5.

### DETAILED DESCRIPTION OF THE EMBODIMENTS

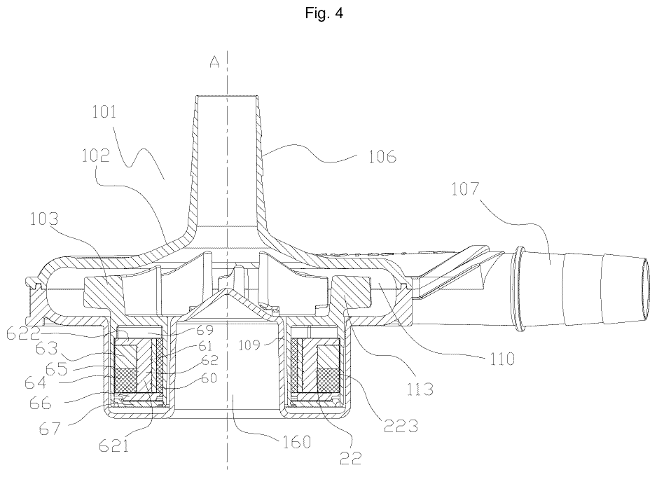

As used herein, the terms “upper”, “lower”, “high”, “low”, “top” and “bottom” are orientations defined when a pump is in the state shown in FIG. 4, and terms “left” and “right” are defined when facing towards the pump with the state shown in FIG. 4.

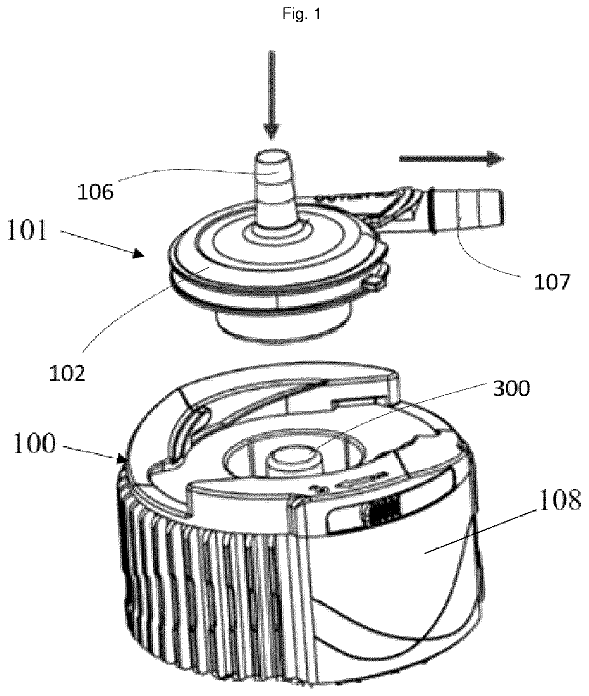

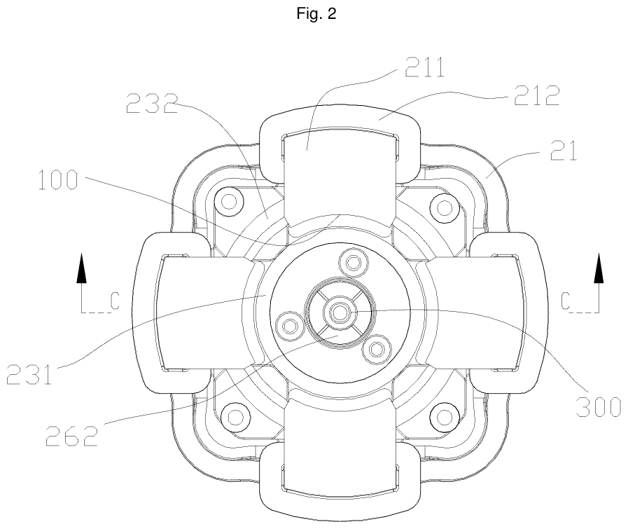

Referring to FIGS. 1-6, an extracorporeal magnetically levitated blood pump of the embodiment includes a motor 100 and a pump head 101 detachably connected with the motor 100. The operative connection between the pump head 101 and the motor 100 adopts the relevant art under the publication numbers CN209187707U, CN209204247U and CN209204246U, which will not be repeated here.

The pump head 101 includes a pump housing 102 and an impeller 113 accommodated in the pump housing 102, and the impeller 113 includes an impeller housing 150 and blades 103 located on the impeller housing 150. The impeller 113 can levitate in the pump housing 102 and can be driven by the motor 100 to rotate around rotation axis A to pump blood from an inlet 106 of the pump housing 102 to an outlet 107. Levitation of the impeller 113 in the pump housing 102 can be realized by a known embodiment provided by CN111561519B or CN112546425B, which will not be repeated here.

The outlet 107 has an outlet flow path leading tangentially into a pump cavity 110, and the outer end of the outlet 107 has a blood outlet. The inlet 106 has an inlet flow path leading into the pump cavity 110 along the axial direction Z, and the upper end of the inlet 106 has a blood inlet. The flow direction (extension direction) of the outlet flow path is perpendicular to the flow direction of the inlet flow path.

The pump housing 102 is made of a plastic material, so as to reduce interference on a magnetically levitated system. As shown in FIG. 4, the pump housing 102 includes an upper cover body and a lower cover body, and the upper cover body fixedly covers the lower cover body to form the pump housing 102. The inlet 106 is located on the upper cover body, and the outlet 107 is mostly located on the lower cover body. The lower cover body provides an upward open structure and is covered by the upper cover body to form the pump cavity 110.

The pump cavity 110 is a flat cavity, and the pump cavity 110 accommodates the blade 103 of the impeller 113. The pump housing 102 further has an annular accommodating cavity 109 accommodating the impeller housing 150, the annular accommodating cavity 109 is located below the pump cavity 110 and communicating with the same, and the annular accommodating cavity 109 and the inlet 106 are respectively located on two sides of axial direction Z of the pump cavity 110. The annular accommodating cavity 109 communicates with the lower side of the pump cavity 110, and the inlet 106 communicates with the upper side of the pump cavity 110.

The extracorporeal magnetically levitated blood pump is a centrifugal magnetically levitated pump, the blades 103 include main blades and shunting blades which are distributed in a staggered manner along the circumferential direction, by designing the shunting blades, the effective circulating area is increased, the flow field is stabilized, further, pump head and pump efficiency of the pump is improved, and better hydraulic performance is provided under low rotating speed. For example, a flow rate of 8 L/min is achieved at 3000 RPM.

The impeller housing 150 and the pump housing 102 form a secondary flow path with a U-shaped (longitudinal) section, and the U-shaped flow paths on two sides of rotation axis A are connected to form a flow path roughly in a W shape. Since damage of blood is from high shear stress and exposure time under the high shear stress, with such design, retention time (also called exposure time, flushing time) of blood in the U-shaped flow path is reduced, then damage to blood by the shear stress in the flow path is reduced, and adverse influence of pumping on blood transport is effectively reduced.

The U-shaped secondary flow path includes an inner annular clearance located at the inner side of the impeller housing 150, a bottom annular clearance located at the bottom side of the impeller housing 150, and an outer annular clearance located on the outer side of the impeller housing 150. The secondary flow path ensures that the complete flushing time is less than 0.5 s.

The driving of the impeller 113 is realized by means of magnetic coupling. Specifically, as shown in FIGS. 1, 2, and 3, an output shaft 300 of the motor 100 is provided with an active magnet 262, and a rotor 155 is disposed in the impeller housing 150 of the impeller 113. As shown in FIG. 4, the rotor 22 includes a passive magnet 60 coupled with the active magnet 262, a rotor permanent magnet 64 coupled to a stator assembly 20, and a balancing ring 62 separated between the passive magnet 60 (inner magnet) and the rotor permanent magnet 64 (outer magnet). The impeller 113 is driven by the motor 100 by virtue of coupling of the active magnet 262 and the passive magnet 60, and levitates in the pump housing 102 by virtue of the coupling effect of the rotor permanent magnet 64 and the stator assembly 20.

The rotor 22 further includes a shielding ring 61 located between the balancing ring 62 and the passive magnet 60, the passive magnet 60 includes a plurality of arc-shaped magnetic monomers, and the plurality of magnetic monomers are fixedly sleeved by the shielding ring 61 to form a whole. The shielding ring 61 is made of a magnetic conductive material such as iron, and is configured to separate the magnetic fields located on two sides and shield the coupling magnetic field from the levitation magnetic field, thus preventing the magnetic fields on two sides from interfering with each other. The magnetic fields on two sides are respectively the inner magnetic field defined by the active and passive magnets 262 and 60 and configured for realizing coupling or driving, and the outer magnetic field defined by the rotor permanent magnet 64 and the stator assembly 20 and configured for realizing levitation.

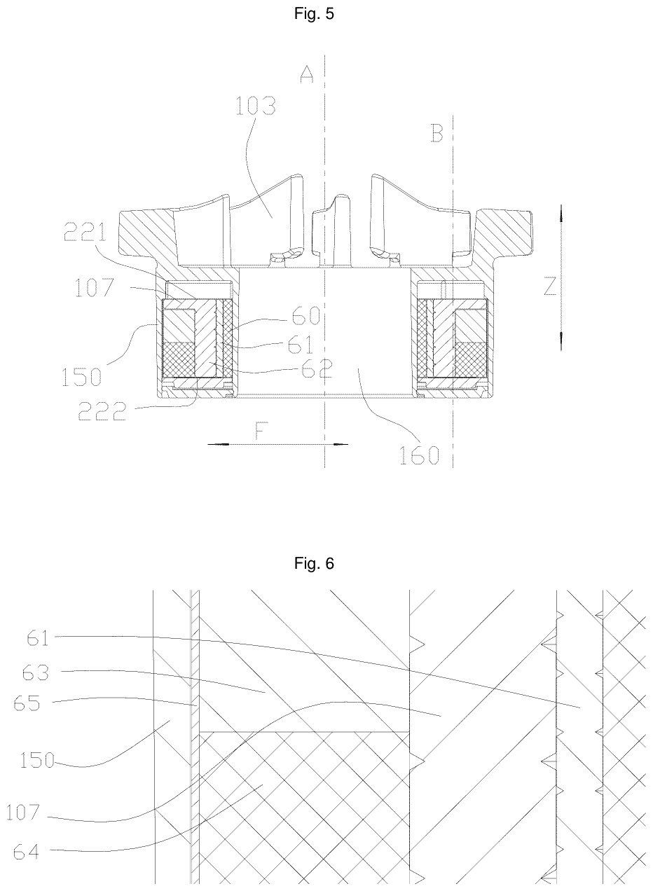

The rotor 22 further includes a deviation correction ring 63 disposed on the outer side of the balancing ring 62, and the deviation correction ring 63 is disposed on the outer side of the balancing ring 62 and is made of a magnetic conductive material. The deviation correction ring 63 is located above the rotor permanent magnet 64. The rotor permanent magnet 64 is fixedly connected to the side of axial direction Z of the deviation correction ring 63, and when facing FIG. 4 or FIG. 5, the rotor permanent magnet 64 is fixedly connected to the lower surface of the deviation correction ring 63. Accordingly, in the radial direction F, the balancing ring 62 is separated among the shielding ring 61, the deviation correction ring 63 and the rotor permanent magnet 64.

Specifically, the passive magnet 60 is bonded to the inner surface of the shielding ring 61, and the outer surface of the shielding ring 61 is bonded to the inner surface of the balancing ring 62. As shown in FIG. 6, to improve the connection strength, glue containing grooves are disposed in inner and outer surfaces of the shielding ring 61, and glue containing grooves are further disposed in inner and outer surfaces of the balancing ring 62. The depth of the glue containing groove of the balancing ring 62 is larger than that of the shielding ring 61. The inner glue containing groove, facing the shielding ring 61, of the balancing ring 62 is staggered with the outer glue containing groove, facing the balancing ring 62, of the shielding ring 61. The rotor permanent magnet 64 and the deviation correction ring 63 arranged up and down (arranged along the axial direction Z) are fixed to the outer surface of the balancing ring 62 by adhesion. The inner and outer surfaces are defined by radial direction.

In order to promote the moment of inertia of the rotor and the stiffness of the rotor 22, the balancing ring 62 has an axial spacing part 621 extending along the axial direction Z and a radial bulge part 622 protruding out of the axial spacing part 621 in the radial direction F. Accordingly, the top surface of the shielding ring 61 and the top surface of the passive magnet 60 are flush with the top surface of the balancing ring 62, and the upper surface 221 of the rotor 22 includes the top surface of the balancing ring 62 (radial bulge part), and the top surfaces of the passive magnet 60 and the shielding ring 61. The outer edge of the radial bulge part 622 is flush with the outer edge of the rotor permanent magnet 64. The axial Z thickness of the radial bulge part 622 is smaller than that of the axial spacing part 621.

For the balancing ring 62 of the embodiment, by increasing one radial bulge part 622, an original straight ring is changed to an L-shaped ring or L-shape-like ring, so that the integral structure of the balancing ring 62 is enlarged, its moment of inertia is increased, the stiffness of the rotor 22 is improved, and then the stiffness of the rotor 22 is improved in a physical or mechanical perspective.

Referring to FIG. 5, on a half longitudinal section, on the side around the rotation axis A, of the rotor 22, a middle line B parallel to the rotation axis A and at a position of a middle circle of the half longitudinal section is defined. The center of gravity of the half longitudinal section is located on the radial outer side of the middle line B. The weight, located on the radial outer side of the middle line B, of the half longitudinal section is larger than that located on the radial inner side of the middle line B. The position of the middle circle is defined as a position of one-half diameter of the sum of outer circle diameter (also referred to as rotor outer diameter) and inner circle diameter (also referred to as rotor inner diameter) of the half longitudinal section.

Meanwhile, for the rotor 22, the weight of the part, between middle circle and outer circle, of the rotor 22 is greater than that of the part, between middle circle and inner circle, of the rotor 22. For example, in an embodiment, the weight of the rotor permanent magnet 64 and the deviation correction ring 63 is greater than that of the shielding ring 61 and the passive magnet 60, so that the center of gravity of the rotor 22 is shifted radially outwards, the moment of inertia is increased, and the stiffness of the rotor is improved.

For the rotor 22 of the embodiment, by disposing the radial bulge part 622, the center of gravity of the half longitudinal section of the rotor 22 is shifted outwards, so that the moment of inertia is improved, and the stiffness of the rotor 22 is improved. Of course, the present disclosure does not exclude the means of improving moment of inertia by improving partial density of the outer side of the middle line B or increasing an accessory part for realizing shift of the center of gravity.

Specifically, the radial bulge part 622 outwards protrudes out of the axial spacing part 621 along the radial direction F, the radial bulge part 622 is disposed at one end of the axial spacing part 621, and the radial bulge part 622 is located on the axial side of the rotor permanent magnet 64.

In the embodiment, the radial bulge part 622 is located above the rotor permanent magnet 64. The radial bulge part 622 is located on the top of the axial spacing part 621. In other embodiments, the radial bulge part 622 also is located at the bottom of the balancing ring 62. The half section (the longitudinal section located on the side of the axis of rotation) of the balancing ring 62 is L-shaped, U (with the opening facing the right)-shaped, or I-shaped. In the embodiment, the half section of the balancing ring 62 is inverted-L-shaped. The balancing ring 62 is of an integrally formed structure of aluminum alloy material, that is, the axial spacing part 621 and the radial bulge part 622 are of an integrally formed structure. The axial spacing part 621 and the radial bulge part 622 are disposed at an included angle other than 180 degrees, and as shown in FIG. 5, the axial spacing part 621 and the radial bulge part 622 are perpendicular, and disposed at an included angle of 90 degrees.

In the embodiment, the rotor 22 includes a sleeve ring 65 sleeving the rotor permanent magnet 64. The sleeve ring 65 extends from the bottom of the rotor 22 to the top of the rotor 22 along axial direction Z. The sleeve ring 65 sleeves the outermost radial side of the rotor 22, and the rotor permanent magnet 64, the deviation correction ring 63, the passive magnet 60, the shielding ring 61 and the balancing ring 62 are sleeved inside the sleeve ring 65. The sleeve ring 65 wraps and fixes other structures (the rotor permanent magnet 64, the deviation correction ring 63, the passive magnet 60, the shielding ring 61 and the balancing ring 62) of the rotor 22 for detecting the position of the rotor 22. The sleeve ring 65 rotates in the stator assembly 20, induction current can be generated inside due to cutting of a magnetic inductance line, an induction magnetic field is further generated due to the induction current, which is detected by sensors (the sensors are disposed between adjacent two stator teeth 211, and the number of the sensors is four) on the outer side, and then detection on the position of the rotor 22 is realized.

The sleeve ring 65 is made of titanium alloy, aluminum alloy or magnesium alloy. In some embodiments, the sleeve ring 65 is made of titanium alloy, so that the wall thickness of the sleeve ring 65 is reduced, and further, a magnetic gap between the rotor 22 and the stator assembly 20 is reduced. In such a case, when current is fixed, the reduced magnetic gap generates a larger magnetic force, so that control force and control efficiency of the motor 100 on the rotor 22 are improved, the stiffness of the rotor 22 is improved, and the stability is improved (the angle of the magnetic field improves the stiffness of the rotor). Specifically, the radial thickness of the sleeve ring 65 is 0.0534-0.1763 mm, further, 0.0728-0.1573 mm, and further, 0.0847-0.1355 mm. For example, the thickness (radial thickness) of the sleeve ring 65 is 0.1 mm.

It is to be noted that any numerical value in the present disclosure includes all values of lower values and upper values in increments of one unit from the lower limit to the upper limit, provided that there is an interval of at least two units between any lower value and any higher value.

For example, the radial thickness of the sleeve ring 65 is 0.0534-0.1763 mm, and further 0.0728-0.1573 mm, for the purpose of illustrating the values such as 0.0535 mm, 0.0538 mm, 0.0611 mm, 0.0734 mm, 0.111 mm, 0.132 mm and 0.1624 mm, which are not explicitly listed above.

As mentioned above, the example range with 0.001 as the interval unit does not exclude increase with an appropriate unit as an interval, such as the numerical units including 0.1, 0.0001, 0.03, 0.004, and 0.5. These are merely examples intended to be clearly expressed, and it can be considered that all possible combinations of numerical values listed between the lowest value and the highest value are explicitly set forth in a similar manner in this specification.

Unless otherwise stated, all ranges include endpoints and all numbers between endpoints. “About” or “approximately” used with a range apply to two endpoints of the range. Therefore, “about 20 to 30” is intended to cover “about 20 to about 30”, at least including specified endpoints.

Other limited explanations about the numerical range appearing herein refer to the above description and will not be repeated here.

Following the above description, the rotor 22 includes a passive magnet 60 coupled to the active magnet 262, and the active magnet 262 and the passive 60 have a coincidence part due to coupling (opposite in radial direction F). In a scene of radial coupling of the active and passive magnets 60, the coincidence part is the height of axial direction Z (also is regarded as axial height). Since coupling of the two is magnetic attraction, once the rotor moves to one side, for example, to the right, the active magnet 262 and the passive magnet 60 approach on the left side and move away on the right side. Therefore, the magnetic attraction force on the left side increases because of the decrease in distance, and the magnetic attraction force on the right side decreases because of the increase in distance. Therefore, the magnetic coupling between the driving magnets (the active magnet 262 and the passive magnet 60) is an adverse factor damaging the stiffness of the rotor. By reducing overall height of the rotor, the coupling area of the active magnet 262 and the passive magnet 60 is reduced, thereby reducing influence of magnetic coupling effect between the active magnet 262 and the passive magnet 60 on stiffness of the rotor, and improving the stiffness of the rotor 22.

Based on the above research, the axial height (overall height) of the rotor of the embodiment is 7.86-10.34 mm, further 8.56-9.87 mm, and further 8.834-9.537 mm. As shown in FIG. 5, the axial height of the rotor 22 is also the axial height of the passive magnet 60. In such a case, the reduction of the overall height of the rotor 22 (decrease of the axial height) reduces the coupling length of the driving magnet 262 (active magnet 262), thereby reducing the adverse impact on levitation stiffness. The balancing ring 62 is provided with the radial bulge part 622, so that the weakening effect of height reduction of the rotor 22 on the moment of inertia is effectively compensated.

In order to facilitate configuration and installation of the L-shaped balancing ring 62 and reduce the overall height of the rotor 22, the length of the rotor permanent magnet 64 along axial direction Z is 2.11-5.87 mm, further, the length of the rotor permanent magnet 64 along axial direction Z is 2.80-5.12 mm, and further, the length of the rotor permanent magnet 64 along axial direction Z is 3.54-4.86 mm.

The length of the deviation correction ring 63 along axial direction Z is 3.67-5.87 mm, further, the length of the deviation correction ring 63 along axial direction Z is 4.35-5.55 mm, and further, the length of the deviation correction ring 63 along axial direction Z is 4.81-5.34 mm. The length of the rotor permanent magnet 64 along axial direction Z is smaller than that of the deviation correction ring 63 along axial direction Z to better hold a plurality of rotor permanent magnets 64. An illustrative example is: the length of the rotor permanent magnet 64 along axial direction Z is 4 mm, and the length of the deviation correction ring 63 along axial direction Z is 5 mm.

Further, in order to adapt to decrease of the overall height of the rotor, avoid loss of moment of inertia and reduce the magnetic gap between the fixed rotors, the radial thickness of the rotor permanent magnet 64 is increased, specifically, the radial thickness of the rotor permanent magnet 64 is 3.15-4.15 mm, further, 3.35-4.0 mm, and further, 3.45-3.84 mm. An illustrative example is: the radial thickness of the rotor permanent magnet 64 is 3.6 mm. The radial thickness of the deviation correction ring 63 is equal to or greater than that of the rotor permanent magnet 64. In the embodiment, the radial thickness of the deviation correction ring 63 is also 3.6 mm. Therefore, in the embodiment, by increasing radial thickness of the rotor permanent magnet 64 and the deviation correction ring 63, reduction of overall height of the rotor 22 is adapted, and meanwhile, the magnetic gap between the fixed rotors is effectively reduced to improve stability of rotation of the rotor.

Relative to the original rotor structure, the overall height (axial height) of the rotor 22 of the embodiment is reduced, so a cushion block 66 is added at the bottom of the rotor 22. By adding the cushion bock 66, the height of the impeller housing 150 does not need to be adaptively reduced, then the impeller housing 150 and the pump housing 102 do not need to be re-designed, meanwhile, reduction of height of the secondary flow path is avoided, so that the hydraulic effect of the extracorporeal magnetically levitated blood pump is guaranteed. Specifically, the impeller housing 150 has an accommodating cavity 69 for accommodating the rotor 22. The accommodating cavity 69 has a lower opening and a bottom cover 67 covering the lower opening. A cushion block 66 is disposed between the rotor 22 and the bottom cover 67. The cushion block 66 is made of plastic and supports the rotor 22 at the bottom of the rotor 22.

To facilitate installation of the fixed rotor 22, the cavity wall of the accommodating cavity 69 is provided with a limiting step (not marked). The rotor 22 is limited between the limiting step and the cushion block 66. The radial width of the limiting step is greater than the radial thickness of the sleeve ring 65, and the sleeve ring 65 is limited between the limiting step and the cushion block 66 in the axial direction Z.

Following the above description, after the pump head 101 is connected to the motor 100, the output shaft 300 of the motor 100 is inserted into the pump housing 102 (specifically, a magnetic coupling cavity 160) of the pump head 101, the active and passive magnets realize coupling through magnetic force, and then the rotation of the motor 100 is transmitted to the impeller 113 to realize rotary driving of the impeller 113.

As shown in FIGS. 1, 2 and 3, the motor 100 includes a motor main body 50, a shaft sleeve 263 is disposed on the output shaft 300 of the motor main body, a plurality of arc-shaped active magnets 262 are disposed outside the shaft sleeve 263, and the plurality of active magnets 262 are fixed by a cylindrical wrapping part 265 (wrapping sleeve 265). The pump housing 102 includes an annular accommodating cavity 109, the impeller 113 includes an impeller housing 150 disposed in the annular accommodating cavity 109 and a plurality of blades 103 disposed on the impeller housing 150. A rotor 22 is disposed in the impeller housing 150, and the rotor 22 includes a plurality of arc-shaped passive magnets 60. After the pump head 101 is connected to the motor 100, the output shaft of the motor 100 is inserted into the pump housing 102 of the pump head 101, the active magnet 262 and the passive magnet 60 realize coupling through magnetic force, and then the rotation of the motor 100 is transmitted to the impeller 113 to realize rotary driving of the impeller 113.

The magnetically levitated motor 100 includes a stator assembly fixed in the motor housing 108. The stator assembly mainly includes an insulation framework 1 and a stator assembly part 20 (also is referred to as a stator core). The stator component body 20 includes a stator yoke 21 in a ring shape and two pairs of stator teeth 211 disposed on the stator yoke 21. The rotor 22 is disc-shaped and rotates around a rotation shaft A. In an ideal situation, the stator 21 and the rotor 22 are coaxial. The stator tooth 211 includes a vertical part 32 and a horizontal part 31 perpendicular to each other, and is in an inverted “L” shape.

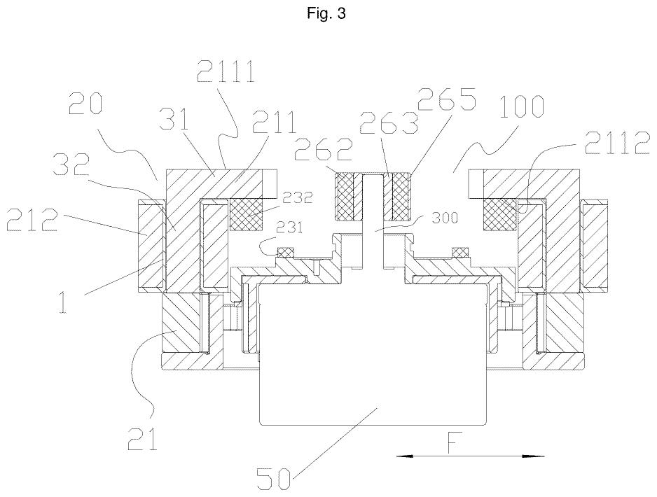

As shown in FIG. 3, a stator permanent magnet 232 is disposed on the lower surface of the horizontal part 31 of the stator tooth 211, and an axial power body 231 located below the stator permanent magnet 232 is disposed on the motor. The stator permanent magnet 232, the axial power body 231 and the rotor permanent magnet 64 form a stiffness gain mechanism of the magnetically levitated bearing, for realizing levitation of the impeller 113. The setting method of the stiffness gain mechanism refers to the present disclosure of CN111561519A, and will not be repeated here.

The deviation correction ring 63 is made of a magnetic conductive material such as iron, in match with the stator assembly 20, is configured to correct the position when the impeller 113 deviates. Specifically, when the rotor 22 is in a balanced position, the stator assembly 20 is in a silent state without power supply. However, once the rotor 22 deviates and is detected by the sensor for detecting the position of the rotor, the controller electrically connected with the stator assembly 20 controls the operation of the stator assembly 20. Namely, a coil 212 winding the vertical part 32 of the stator tooth 211 is powered on to generate an induction magnetic field, the induction magnetic field forms a magnetic loop between the opposite stator tooth 211 and deviation correction ring 63 through the stator yoke, thus two opposite stator teeth 211 form two magnetic force effects in the same direction on the rotor, the rotor 22 is pulled back to a balanced position, and deviation correction on the position of the rotor 22 is realized.

As shown in FIGS. 3-5, the rotor 22 includes an upper surface 221, a lower surface 222, and a circumferential outer surface 223. In an ideal situation, the upper surface 2111 of the horizontal part 31 of the stator tooth 211 is flush with the upper surface 221 of the rotor 22, and the lower surface 2112 of the horizontal part 31 of the stator tooth 211 is flush with the lower surface 222 of the rotor 22. Meanwhile, there is a magnetic gap with circumferentially equal width between the inner circumferential surface 2113 of the horizontal part 31 of the stator tooth 211 and the outer circumferential surface 223 of the rotor 22 (outer surface 223 of a metal ring). Specifically, the magnetic gap between the stator assembly 20 and the rotor 22 is 1.12-3.2 mm, further 1.57-2.83 mm, and further, 1.711-2.566 mm. In such a case, the reduced magnetic gap generates a larger magnetic force under the condition that current is fixed, so that control force and control efficiency of the motor on the rotor are improved, the stiffness of the rotor is increased, and the stability is good.

It should be understood that the above description is for illustrative purposes and not for limitation purposes. By reading the above description, numerous implementation modes and numerous applications beyond the examples provided will be apparent to those skilled in the art. Therefore, the scope should not be determined by reference to the above description, but by reference to the full scope of the attached claims and their equivalents. For the comprehensive purpose, all articles and references, including present disclosure of the patent applications and announcements are incorporate by reference herein. The omission of any aspect of the subject matter disclosed herein in the forgoing claim is not intended to waive the subject matter, or should it be considered that the inventor has not considered the subject matter as a part of the disclosed subject matter.

What is claimed is:

1. An extracorporeal magnetically levitated blood pump, comprising:
a motor;
a pump head operably connected to the motor, comprising:
a pump housing, with an inlet and an outlet;
an impeller, which is accommodated in the pump housing and is configured to levitate in the pump housing and driven by the motor to rotate, so that blood is pumped from the inlet to the outlet; wherein the impeller comprises: an impeller housing and blades disposed on the impeller housing; a rotor is disposed in the impeller housing, and an axial height of the rotor is 7.86-10.34 mm.

2. The extracorporeal magnetically levitated blood pump as claimed in claim 1, wherein the axial height of the rotor is 8.56-9.87 mm.

3. The extracorporeal magnetically levitated blood pump as claimed in claim 1, wherein the axial height of the rotor is 8.834-9.537 mm.

4. The extracorporeal magnetically levitated blood pump as claimed in claim 1, wherein on a half longitudinal section, on a side around a rotation axis, of the rotor, a middle line parallel to the rotation axis and at a position of a middle circle of the half longitudinal section is defined; a center of gravity of the half longitudinal section is located on a radial outer side of the middle line, and the position of the middle circle is defined as a position of one-half diameter of a sum of outer circle diameter and inner circle diameter of the half longitudinal section.

5. The extracorporeal magnetically levitated blood pump as claimed in claim 4, wherein a weight, located on the radial outer side of the middle line, of the half longitudinal section is larger than a weight, located on a radial inner side of the middle line; or a weight of a part, between the middle circle and the outer circle, of the rotor is greater than a weight of a part, between the middle circle and the inner circle, of the rotor.

6. The extracorporeal magnetically levitated blood pump as claimed in claim 1, wherein the motor comprises an active magnet disposed on a motor shaft and a stator assembly disposed in a motor housing; the rotor comprises: a passive magnet coupled with the active magnet, a rotor permanent magnet located on an outer side of the passive magnet and coupled with the stator assembly, and a balancing ring; the impeller is driven by the motor by virtue of coupling of the active magnet and the passive magnet, and levitates in the pump housing by virtue of the coupling effect of the rotor permanent magnet and the stator assembly;
the balancing ring has an axial spacing part extending along an axial direction of the balancing ring and located between the passive magnet and the rotor permanent magnet and a radial bulge part protruding out of the axial spacing part in a radial direction of the balancing ring.

7. The extracorporeal magnetically levitated blood pump as claimed in claim 6, wherein the radial bulge part is located at an end part of the axial spacing part, extends radially outward perpendicular to the axial spacing part, and is located on an axial side of the rotor permanent magnet.

8. The extracorporeal magnetically levitated blood pump as claimed in claim 6, wherein an outer edge of the radial bulge part is flush with an outer edge of the rotor permanent magnet.

9. The extracorporeal magnetically levitated blood pump as claimed in claim 1, wherein the rotor further comprises a sleeve ring sleeving outside the rotor permanent magnet, and a radial thickness of the sleeve ring is 0.0534-0.1763 mm.

10. The extracorporeal magnetically levitated blood pump as claimed in claim 9, wherein the axial thickness of the sleeve ring is 0.0728-0.1573 mm; or the axial thickness of the sleeve ring is 0.0847-0.1355 mm.

11. (canceled)

12. The extracorporeal magnetically levitated blood pump as claimed in claim 9, wherein the sleeve ring is made of titanium alloy, aluminum alloy or magnesium alloy.

13. The extracorporeal magnetically levitated blood pump as claimed in claim 6, wherein an axial height of the rotor permanent magnet is 2.11-5.87 mm.

14. The extracorporeal magnetically levitated blood pump as claimed in claim 6, wherein an axial height of the rotor permanent magnet is 2.80-5.12 mm.

15. The extracorporeal magnetically levitated blood pump as claimed in claim 6, wherein an axial height of the rotor permanent magnet is 3.54-4.86 mm.

16. The extracorporeal magnetically levitated blood pump as claimed in claim 6, wherein the rotor further comprises a deviation correction ring disposed on an outer side of the balancing ring and made of a magnetic conductive material, and an axial height of the deviation correction ring is 3.67-5.87 mm.

17. The extracorporeal magnetically levitated blood pump as claimed in claim 16, wherein the axial height of the deviation correction ring is 4.35-5.55 mm, or the axial height of the deviation correction ring is 4.81-5.34 mm.

18. (canceled)

19. The extracorporeal magnetically levitated blood pump as claimed in claim 13, wherein a radial thickness of the rotor permanent magnet is 3.15-4.15 mm; or a radial thickness of the rotor permanent magnet is 3.35-4.0 mm; or a radial thickness of the rotor permanent magnet is 3.45-3.84 mm.

20. (canceled)

21. (canceled)

22. The extracorporeal magnetically levitated blood pump as claimed in claim 6, wherein the rotor further comprises a shielding ring located between the balancing ring and the passive magnet and made of a magnetic conductive material, and a top surface of the shielding ring and a top surface of the passive magnet are flush with a top surface of the balancing ring.

23. The extracorporeal magnetically levitated blood pump as claimed in claim 1, wherein the impeller housing has an accommodating cavity for accommodating the rotor, a bottom of the accommodating cavity is covered by a bottom cover, and a cushion block is disposed between the rotor and the bottom cover.

24. The extracorporeal magnetically levitated blood pump as claimed in claim 6, wherein the stator assembly comprises a stator permanent magnet, and a magnetic gap between the rotor and the stator permanent magnet is 1.12-3.2 mm, or the magnetic gap is 1.57-2.83 mm, or the magnetic gap is 1.711-2.566 mm.

25. (canceled)

26. (canceled)
